(19) United States
(12) Patent Application Publication
(10) Pub. No.: US 2012/0201981 A1
(43) Pub. Date: Aug. 9, 2012

(54) COMBINATIONS OF BIOLOGICAL CONTROL AGENTS WITH A NEMATICIDAL SEED COATING

(75) Inventor: Jorn Ole BECKER, Riverside, CA (US)

(73) Assignee: The Regents of the University of California, Oakland, CA (US)

(21) Appl. No.: 13/452,609

(22) Filed: Apr. 20, 2012

Related U.S. Application Data
(63) Continuation of application No. 12/305,564, filed on Oct. 16, 2009.
(60) Provisional application No. 60/815,197, filed on Jun. 19, 2006.

(51) Int. Cl. A01N 63/00; A01P 5/00; B32B 1/06; A01P 7/02; A01P 9/00; A01H 5/00; A01C 1/06; A01P 7/04

(52) U.S. Cl. 428/34.1; 424/93.46; 504/100; 800/298

(57) ABSTRACT

This invention provides combinations of at least one biological control agent and at least one nematicide to enhance plant protection against pests and pathogens.

This application is a continuation of U.S. patent application Ser. No. 12/305,564, filed Oct. 16, 2009, which is the National Stage of International Application No. PCT/US2007/071467, filed Jun. 18, 2007, which claims the benefit U.S. provisional application No. 60/815,197, filed Jun. 19, 2006, each of which is herein incorporated by reference in its entirety for all purposes.

Phytoparasitic nematodes lead to severe plant production constraints in many agronomic and horticultural crops. Severe infestations with endoparasitic nematodes such as certain root-knot or cyst nematodes can result in yield losses of 10% to 50%. Worldwide crop losses due to plant parasitic nematodes have been estimated at $80 billion annually.

Current pest management options for controlling nematodes are very limited. Soil fumigants and effective non-fumigant nematicides, especially carbamate and organophosphate compounds, are increasingly under regulatory pressure because of potential undesirable effects on users, consumers, and the environment. Other effective methods to reduce plant-parasitic nematode populations, such as exposing infested soil to heat by steam treatment, are technically difficult and too costly for field uses.

Certain seed treatments have significant activity against plant-parasitic nematodes. For example, abamectin seed treatment has been shown to effectively protect roots of young seedlings against various plant pests, including plant-parasitic nematodes. Non-protected root systems show stunting, and in case of root-knot nematodes (Meloidogyne spp.) show more severe galling, in comparison to abamectin-protected plants. These below-ground differences are reflected in significant height and dry weight differences of the shoots. However, seed treatment protection against nematode invasion often lasts for only a relatively short period of time. It is therefore desirable to develop a treatment that is capable of extending the protection period, e.g., for use with long season crops and in climates where multiple generations of pests, e.g., nematodes, occur.

Biological control of plant-parasitic nematodes and other pests has been suggested as a potential alternative to chemical management (see, e.g., Kerry, 1987 Biological Control. In: Principles and practice of nematode control in crops, R. H. Brown and B. R. Kerry, eds., pp. 233-263, Academic Press, London., 1987; and Stirling, Biological control of plant parasitic nematodes. CAB International, Wallingford, UK, 1991). Nematophagous fungi are of particular interest for this application. Nematophagous fungi are generally divided into two categories: a) nematode-trapping fungi that produce mechanical or adhesive traps, and b) endoparasitic fungi which infect namatodes by hyphal penetration or when their conidia (spores) are ingested or adhere to the cuticle of the nematodes. In the past, attempts to employ nematophagous fungi in non-sterile soil have been largely ineffective. The few products that are commercially available on the international market have generally poor performance records.

More recently, the focus of research has shifted from trapping fungi to the female -and egg-parasitizing fungi. These fungi are either obligate parasites of nematodes or facultative predators with the ability to colonize root surfaces and epidermal/cortical tissues of roots, but do not cause obvious damage to the plant. Their target hosts include the economically most important root-knot nematodes (Meloidogyne spp.), and cyst nematodes (Heterodera spp., Globodera spp.). Attempts using these fungi as potential biological control organisms are also well documented (e.g., Kerry, B. R. Journal of Nematology 22:621-631, 1990; Stirling, 1991, supra; and Jaffee, B. A. Canadian Journal of Microbiology 38:359-364, 1992). However, the results were often disappointing, as these fungi typically failed to protect the young seedling roots against the invading second-stage juveniles of endoparasitic nematodes.

In view of the foregoing, there is a need for improved methods of controlling nematodes and other plant pests and pathogens.

An embodiment of the invention includes methods and combination treatments relating to enhancing protection of plants against pests/pathogens and improving the health of plants. The methods may be used on any plants, but in some embodiments, the methods may be particularly useful for treating nursery plant or plants grown in a container, e.g., prior to transplantation.

In one aspect, the invention comprises methods of treating a plant with a combination treatment comprising one or more of a nematicide, such as an avermectin, and one or more of a biocontrol agent. Thus, in one embodiment, the invention includes a method of enhancing pest resistance in a plant, the method comprising applying a pesticide composition comprising a nematicide, such as an avermectin, for example and not for limitation, abamectin, to a plant propagation material, such as a seed; and applying at least one biocontrol agent. The biocontrol agent may be a nematode-antagonistic biocontrol agent.

100101 An embodiment of the present invention also relates to a method that comprises (i) treating a plant propagation material, such as a seed, with one or more of a nematicide, (ii) applying one or more of a biocontrol agent to the locus of the plant propagation material, often before step (iii), (iii) planting or sowing the treated propagation material, and (iv) achieving enhancement of pest resistance of the treated plant propagation material, parts of plant and/or plant grown from the treated propagation material.

In some embodiments, the step of applying the biocontrol agent comprises inoculating the soil or planting media in which the plant propagation material is planted (or to be planted) with the biocontrol agent. This step of inoculating can be performed prior to planting, while planting the propagation material, or after planting the propagation material.

The step of applying the biocontrol agent can comprise treating the soil or planting media into which plant propagation material, such as a seed, is sown with the biocontrol agent prior to, or at the same time as, planting. In other embodiments, the step of applying the biocontrol agent to the propagation material may, for example, comprise treating the propagation material with the biocontrol agent. A seed that has been treated with biocontrol agent may also have a treatment comprising an additional pesticidal composition.

In some embodiments, the step of applying the pesticide composition to the plant propagation material, such as a seed, comprises applying the pesticide composition to the soil or planting media in which the plant propagation material is planted. Such a treatment may take place at any time in the planting process, including prior to planting the propagation material, as the propagation material is being planted, or after planting the propagation material; and may be applied one or more times.

In some embodiments, the step of applying the pesticide composition to the plant propagation material comprises treating the plant propagation material, such as a seed, with the pesticide composition, preferably before plant propagation material, such as a seed, is sowed or planted.

At least one biocontrol agent can be used in the invention. In various embodiments, the biocontrol agent can be selected from one or more of a fungus, bacteria, or other agent. Often, anti-nematode bacteria or anti-nematode fungal biocontrol agents are used. In particular embodiments, the biocontrol agents can be an endoparasitic fungus, e.g., a member selected from Chytridiomycetes, Oomycetes, Zygomycetes, Deuteromycetes, and Basidiomycetes.

In other embodiments of the invention, the anti-nematode biocontrol fungus can be a member of a genus selected from Catenaria, Myrothesium, Myzocytium, Bacillus, Haptoglossa, Meristacrum, Dactylella, Paecilomyces, Cephalosporium, Meria, Harposporium, Nematoctonus, Rhopalomyces, Verticillium, Pochonia, Saprolegnia, Cylindrocarpon, Nematophthora, Hirsutella, and Monoacrosporium. As a non-limiting example, the biocontrol agent can be Pochonia chlamydosporia (syn. Verticillium chlamydosporium), Myrothesium verrucaria, Dactylella oviparasitica, Fusarium oxysporum, Paecilomyces lilacinus, Plectosphaerella cucumerina, Hirsutella rhossiliensis, Drechmeria coniospora, Myzocytium spp., Lagenidium spp., Catenaria nguillulae, Nematophora gynophila and others.

The invention also provides embodiments in which the biocontrol agent can be a bacterial species, such as, but not limited to, a rhizobacterial species or a species associated with entomopathogenic nematodes. In particular embodiments, the biocontrol agent can be a species selected from Pasteuria spp., Pseudomonas spp., Bacillus spp., Corynebacterium, Agrobacterium spp., and Paenibacillus spp. As a non-limiting example, the bacterial biological control agents can be endoparasitic bacterium of the genus Pasteuria, e.g. Pasteuria penetrans, Baccilus firm us, Pseudomonas cepacia, Corynebacterium paurometabolum, P. thornei, P. nishizawae, Candidatus Pasteuria usgae sp. nov., or Candidatus Pasteuria sp. strain HG.

In some embodiments of the invention, the methods may further comprise applying a second biocontrol agent. The second biocontrol agent can be a different type of biocontrol agent. For example, and not for limitation, if a first biocontrol agent is a bacterial agent, the second biocontrol agent can be a fungus; or it can be the same type of biocontrol agent, but from a different class, genus, species, or strain, e.g., both the first and second biocontrol agent can be fungi, but can be a different species. The second biocontrol agent can be applied at the same time as the first application of one or more nematicide and one or more first biocontrol agent, or it can be applied before or after the combination treatment.

In some methods of the invention, such as but not limited to those methods in which the first biocontrol agent can be an endoparasitic fungus, a second biocontrol agent can also be an endoparasitic fungus that is different from the first.

The invention can also comprise a method where the pesticide composition contains additional pesticidal agents as mixing partners. For example and not for limitation, at least one additional insecticide, nematicide, acaricide or molluscicide can be mixed with the pesticide composition. Such additional pesticidal agents can be selected, for example, from cyanoimine acetamiprid, nitromethylene nitenpyram, clothianidin, dinotefuran, fipronil, lufenuron, pyripfoxyfen, thiacloprid, fluxofenime; imidacloprid, thiamethoxam, beta cyfluthrin, fenoxycarb, lamda cyhalothrin, diafenthiuron, pymetrozine, diazinon, disulphoton, profenofos, furathiocarb, cyromazin, cypermethrin, tau-fluvalinate, tefluthrin, Bacillus thuringiensis products, and chlorantraniliprole.

In some embodiments, the pesticide composition used in a method of the invention can additionally be mixed with at least one fungicide that is selected from azoxystrobin, difenoconazole, fludioxonil, fluoxastrobin, metalaxyl, R-metalaxyl, mefenoxam, myclobutanil, captan, orysastrobin, enestrobin, thiabendazole, thiram, acibenzolar s-methyl, trifloxystrobin, a compound of formula A and a compound of formula B or a tautomer of each compound represented below.

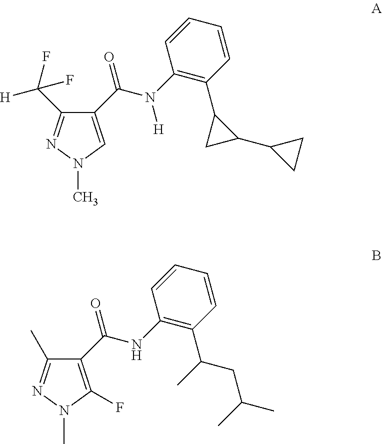

Such a fungicide can be selected such that when a biocontrol agent that is a fungus is included in the treatment, the biocontrol fungus is resistant to the fungicide.

Especially preferred mixing partners are metalaxyl, metalaxyl-M, thiamethoxam, difenoconazole, fludioxonil, azoxystrobin, trifloxystrobin, acibenzolor s-methyl, silthiofam, tefluthrin, imidacloprid, clothianidin, myclobutanil and thiabendazole.

In another embodiment, the invention provides combination compositions for enhancing pest resistance in plants. Thus, the invention also provides a combination composition comprising a pesticide agent comprising an effective amount of one or more of a nematicide, such as an avermectin, e.g., abamectin, and an effective amount of at least one biocontrol agent, e.g., an anti-nematode biocontrol agent.

The combination compositions of the invention can also comprise at least one additional insecticide, nematicide, acaricide or molluscicide, for example and not for limitation, cyanoimine, acetamiprid, nitromethylene nitenpyram, clothianidin, dinotefuran, fipronil, lufenuron, pyripfoxyfen, thiacloprid, fluxofenime; imidacloprid, thiamethoxam, beta cyfluthrin, fenoxycarb, lamda cyhalothrin, diafenthiuron, pymetrozine, diazinon, disulphoton; profenofos, furathiocarb, cyromazin, cypermethrin, tau-fluvalinate, chlorantraniliprole (Rynaxapyr), tefluthrin, and Bacillus thuringiensis products.

In additional embodiments, a combination composition of the invention can further comprise at least one additional fungicide, such as azoxystrobin, orysastrobin, enestrobin, difenoconazole, fludioxonil, fluoxastrobin, metalaxyl, R-metalaxyl, mefenoxam, myclobutanil, thiabendazole, trifloxystrobin, a compound of formula A or a compound of formula B, as provided above. Such a fungicide is selected such that a fungal biocontrol agent that may be present in a composition of the invention is resistant to the fungicide.

In particular embodiments, the at least one biocontrol agent included in a composition can be an endoparasitic fungus, or a member of a genus selected from Catenaria, Myzocytium, Haptoglossa, Meristacrum, Dactylella, Paecilomyces, Cephalosporium, Meria, Harposporium, Nematoctonus, Rhopalomyces, Verticillium, Pochonia, Saprolegnia, Cylindrocarpon, Nematophthora, Hirsutella, Myrothecium, and Monoacrosporium. In particular embodiments, the at least one biocontrol fungus present in a composition of the invention is Pochonia chlamydosporia.

In other embodiments, the at least one biocontrol agent can be a bacterial agent, for example and not for limitation, a rhizobacteria, or a member of a genus selected from Pasteuria, Pseudomonas, Corynebacterium, and Bacillus.

The combination compositions of the invention can also comprise a second biocontrol agent, where the second biocontrol agent can be the same type of agent as the first, but it can be from a different genus, species or strain. In other embodiments, the first and second biocontrol agents can be different types of agents. In particular embodiments, the combination can comprise at least two anti-nematode biocontrol agents, for example and not for limitation, two anti-nematode fungal biocontrol agents. As a non-limiting example, the two anti-nematode fungal biocontrol agents can be two endoparasitic fungi.

In other embodiments, a second biocontrol agent can be a bacterial agent. The second agent can be used either with another bacterial biocontrol agent or with a different type of biocontrol agent, such as but not limited to a fungus.

The invention also provides nematicide/biocontrol agent plant propagation material compositions, such as an avermectin/biocontrol agent plant propagation material composition, in which a nematicide/biocontrol agent combination composition further comprises a plant propagation material, such as a seed. Typical embodiments of the invention include compositions that comprise an abamectin-treated plant propagation material, e.g., a seed, and at least one biocontrol agent. In particular embodiments, a seed treatment can comprise both abamectin and a biocontrol agent. In this respect, the plant propagation material has adhered thereto a nematicide and a biocontrol agent. Accordingly, the present invention also provides a plant propagation material treated with the composition comprising one or more of a nematicide and one or more of a biocontrol agent.

In still other embodiments, plant propagation material compositions of the invention can additionally comprise soil or other planting media, which may be inoculated with one or more biocontrol agents, and a container, e.g., that is suitable for growing a plant in a nursery or a plant that is to be transplanted. In this respect, the present invention makes available a container having therein an amount of soil in which a plant or a part of a plant is grown from a treated plant propagation material, wherein the plant propagation material of the plant, e.g. seed, is treated with a pesticidal composition comprising one or more of a nematicide and either (i) the seed is also treated with one or more a biological agent or one or more of a biological agent is applied to the soil or (ii) both the seed is treated and soil applied with the same or different biological agent(s).

In another aspect, the invention provides a method for improving the growth of a plant, comprising (i) applying a composition that comprises one or more of a nematicide, such as an avermectin, e.g., abamectin, to a plant propagation material, such as a seed, (ii) applying one or more of a biocontrol agent to either the plant propagation material or locus thereof, (iii) planting or sowing the treated plant propagation material, (iv) allowing the treated plant propagation material to germinate and (v) transplanting the young plant to another site, such another container or open soil bed.

Accordingly, the invention provides a method for improving the transplant health of a plant, comprising applying to a plant, plant propagation material, e.g., a seed, or part of a plant that is to be transplanted at some stage after initial planting, or to a locus thereof, a combination that comprises one or more of a nematicide, such as an avermectin, e.g., abamectin, and one or more of a biocontrol agent. Such treatment methods can be performed in accordance with the embodiments of the methods for treating a plant to enhance resistance to pests, as described above.

### DRAWING DESCRIPTION

FIG. 1 provides a summary of exemplary data from a trial showing plant growth responses to single and combination treatments with abamectin and a biological control agent. Legend: diagonal lines, 3 week height; cross-hatched lines, 8 week vine length.

### FURTHER DESCRIPTION OF INVENTION

The term “biocontrol agent” refers to an organism that inhibits or reduces plant infestation and/or growth of plant pathogens, such as pathogenic fungi, bacteria, and nematodes, as well as arthropod pests such as insects, arachnids, chilopods, diplopods, or that inhibits plant infestation and/or growth of a combination of plant pathogens.

The term “nematode-antagonistic biocontrol agent” as used herein refers to an organism that inhibits nematode activity, growth or reproduction, or reduces nematode disease in plants.

“Inhibition of nematode growth” refers to any aspect by which nematode disease in a plant is reduced, including, but not limited to, slowing nematode growth; reducing reproduction, hatching, mate and host-finding; and killing nematodes.

The term “nematicide” refers to a compound having an effect on, such as reduction in the damage caused by, agricultural-related nematodes. Examples include an avermectin (e.g., abamectin), carbamate nematic ides (e.g., aldicarb, thiadicarb, carbofuran, carbosulfan, oxamyl, aldoxycarb, ethoprop, methomyl, benomyl, alanycarb), organophosphorus nematicides (e.g., phenamiphos (fenamiphos), fensulfothion, terbufos, fosthiazate, dimethoate, phosphocarb, dichlofenthion, isamidofos, fosthietan, isazofos ethoprophos, cadusafos, terbufos, chlorpyrifos, dichlofenthion, heterophos, isamidofos, mecarphon, phorate, thionazin, triazophos, diamidafos, fosthietan, phosphamidon), and certain fungicides, such as captan, thiophanate-methyl and thiabendazole. Also included as a nematicide is a compound of formula X,

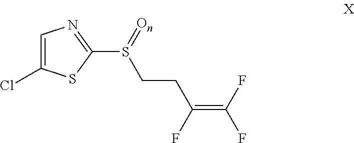

wherein n is 0, 1 or 2 and the thiazole ring may be optionally substituted. Abamectin, aldicarb, thiadicarb, dimethoate, methomyl, a compound of formula X and oxamyl are preferred nematicides for use in this invention.

The term “avermectin” refer to any of the members of the avermectin class of compounds, which are disclosed as milbemycins and avermectins, for example, in U.S. Pat. Nos. 4,310,519; and 4,427,663. Avermectins are known to the person skilled in the art. They are a group of structurally closely related pesticidally active compounds that are obtained by fermentation of a strain of the microorganism Streptomyces avermitilis. Derivatives of avermectins can be obtained via conventional chemical syntheses. “Abamectin” is a mixture of avermectin Bᵢₐ and avermectin B_{ib} and is described, for example, in The Pesticide Manual, 10.sup.th Ed. (1994), The British Crop Protection Council, London, page 3. The designation “abamectin” and “avermectin” include derivatives. Acceptable avermectins useful in the invention include, for example, ivermectin, doramectin, selamectin, emamectin, and abamectin.

The term “plant propagation material” is understood to denote all the generative parts of the plant, such as seeds, which can be used for the multiplication of the plant, and vegetative plant material such as cuttings and tubers (for example, potatoes, sugar cane). Thus, reference may be made, e.g., to the seeds (in the strict sense), roots, fruits, tubers, bulbs, rhizomes, or other parts of plants. Germinated plants and young plants, e.g., which are to be transplanted after germination or after emergence from the soil, may also be referred to as plant propagation material. These young plants may also be protected before transplantation by a total or partial treatment by immersion of the plant propagation material with the composition described herein.

Parts of plant and plant organs that grow at a later time are any sections of a plant that develop from a plant propagation material, such as a seed. Parts of plant, plant organs, and plants can also benefit from the pathogenic and/or pest damage protection achieved by the application of the combination treatment of the invention on to the plant propagation material. In an embodiment, certain parts of plant and certain plant organs that grow at later point in time can also be considered as plant propagation material, which can themselves be applied (or treated) with the combination; and consequently, the plant, further parts of the plant and further plant organs that develop from the treated parts of plant and treated plant organs can also benefit from the pathogenic and/or pest damage protection achieved by the application of the combination treatment on to the certain parts of plant and certain plant organs

The term “applying a pesticide composition” refers to any method of treating a plant, a part of a plant, or soil, or other planting media in which a plant is planted (or is to be planted) with an agent that inhibits pest infestation of a plant and/or pest growth, or an agent that limits disease in a plant due to pests or pathogens.

Methods for applying or treating pesticidal active ingredient compositions and mixtures thereof on to plant propagation material, especially seeds, are known in the art, and include dressing, coating, pelleting and soaking application methods of the propagation material.

The active ingredients can be applied to the seeds using conventional treating techniques and machines, such as fluidized bed techniques, the roller mill method, rotostatic seed treaters, and drum coaters. Other methods, such as spouted beds may also be useful. The seeds may be pre-sized before coating. After coating, the seeds are typically dried and then transferred to a sizing machine for sizing. Such sizing and treating procedures are known in the art.

In one embodiment, the combination can be applied or treated on to the plant propagation material by a method such that the germination is not induced; generally seed soaking induces germination because the moisture content of the resulting seed is too high. Accordingly, examples of suitable methods for applying (or treating) plant propagation material, such as a seed, are seed dressing, seed coating or seed pelleting and the like.

In a typical embodiment, the plant propagation material is seed. Although it is believed that the present method can be applied to a seed in any physiological state, it is preferred that the seed be in a sufficiently durable state that it incurs no damage during the treatment process. Typically, the seed would be a seed that had been harvested from the field; removed from the plant; and separated from any cob, stalk, outer husk, and surrounding pulp or other non-seed plant material. The seed would preferably also be biologically stable to the extent that the treatment would cause no biological damage to the seed. It is believed that the treatment can be applied to the seed at any time between harvest of the seed and sowing of the seed or during the sowing process (seed directed applications). The seed may also be primed according to techniques understood by those skilled in the art either before or after the treatment.

Even distribution of the active ingredients and adherence thereof to the seeds is desired during propagation material treatment. Treatment could vary from a thin film (dressing) of the formulation containing the active ingredient(s) on a plant propagation material, such as a seed, where the original size and/or shape are recognizable to an intermediary state (such as a coating) and then to a thicker film (such as pelleting) with many layers of different materials (such as carriers, for example, clays; different formulations, such as of other active ingredients; polymers; and colourants) where the original shape and/or size of the seed is no longer recognizable.

The seed treatment occurs to an unsown seed. The term “unsown seed” is meant to include seed at any period between the harvest of the seed and the sowing of the seed in the ground for the purpose of germination and growth of the plant.

Treatment to an unsown seed is not meant to include those practices in which the active ingredient is applied to the soil, but would include any application practice that would target the seed during the planting process.

Preferably, treatment occurs before sowing of the seed so that the sown seed has been pre-treated with the combination treatment of the invention. In particular, seed coating or seed pelleting are preferred in the treatment of the combinations described herein. As a result of the treatment, the active ingredients in the combination are adhered on to the surface of the seed and therefore available for pest and/or disease control.

The treated seeds can be stored, handled, sowed and tilled in the same manner as any other active ingredient treated seed.

Methods of applying pesticidal compositions to the soil can be via any suitable method which ensures that the agents penetrate the soil. For example and not for limitation, nursery tray application, in furrow application, soil drenching, soil injection, drip irrigation, application through sprinklers or central pivot, incorporation into soil (broad cast or in band) are included in such suitable methods.

The term “inoculating the soil” as used herein refers to a process of adding spores or some part of a biocontrol organism to the planting substrate. The process of inoculating the soil does not imply that the biocontrol agent is already active, but it simply means that some part of the organism has been placed in the planting medium.

The term “resistant” in the context of the resistance of a biocontrol agent to a pesticide, e.g., a fungicide, refers to the ability of the resistant biocontrol agent to grow and/or multiply or remain metabolically active in the presence of the pesticide. As used herein, an agent is “resistant” when it is immune to the activity of the pesticide.

The term “improving the transplant health” of a plant refers to increasing the ability of a plant to grow following transplantation in comparison to a plant that has not been treated with a combination treatment of the invention. Any number of endpoints reflects an increased ability of a plant to grow, including improvements in the appearance of a plant as well as actual measurements of plant growth, such as plant height, etc. The improvement in the growing (or growth) characteristics of a plant, such as reflected in improved transplant health, is indicated by improvements in one or more observed plant traits as compared to untreated plants. It can, for example, manifest in improving the yield and/or vigor of the plant or quality of the harvested product from the plant, which improvement may not be connected to the control of diseases and/or pests. Examples of enhanced plant traits include, but are not limited to, increased stem girth, early flowering, synchronized flowering, decreased lodging, delaying or eliminating tie-up of crops, increased disease resistance, enhanced water utilization, including but not limited to decreased watering and/or less frequent watering, higher yield, higher quality/healthier plant appearance, including but not limited to better color, greater transportability, decreased insect damage, and smaller plant canopies.

“Enhancing pest resistance in a plant” refers to improving the growth characteristics and/or yield, and/or disease incidence in a plant that is treated with a combination treatment of the invention in comparison to a plant that is untreated.

As used herein the phrase “improving the yield” of a plant relates to an increase in the yield of a product of the plant by a measurable amount over the yield of the same product of the plant produced under the same conditions, but without the application of the subject method. It is preferred that the yield be increased by at least about 0.5%, more preferred that the increase be at least about 1%, even more preferred is about 2%, and yet more preferred is about 4%, or more. Yield can be expressed in terms of an amount by weight or volume of a product of the plant on some basis. The basis can be expressed in terms of time, growing area, weight of plants produced, amount of a raw material used, or the like.

As used herein the phrase “improving the vigor” of a plant relates to an increase or improvement of the vigor rating, or the stand (the number of plants per unit of area), or the plant height, or the plant canopy, or the visual appearance (such as greener leaf color), or the root rating, or emergence, or protein content, or increased tillering, or bigger leaf blade, or less dead basal leaves, or stronger tillers, or less fertilizer needed, or less seeds needed, or more productive tillers, or earlier flowering, or early grain maturity, or less plant verse (lodging), or increased shoot growth, or earlier germination, or any combination of these factors, or any other advantages familiar to a person skilled in the art, by a measurable or noticeable amount over the same factor of the plant produced under the same conditions, but without the application of the subject method.

Accordingly, the present invention also provides a method of improving the growing characteristics of a plant by the method steps defined herein.

The terms “planting media” or “media” or “growth media” as used herein refer to any media that can support plant growth. The term includes soil, as well as media such as rock, wool, vermiculite, etc. The terms “soil” or “plant environment” for plants in the practice of the method of the present invention mean a support for use in culture of a plant and especially a support in which roots are to be grown. The terms are not limited in material quality, but include any material that may be used so far as a plant can be grown therein. For instance, so-called various soils, seedling mat, tapes, water or hydroponic solutions and the like can also be used. Specific examples of the material constituting the soil or cultivation carrier include, without limitation, sand, peat moss, perlite, vermiculite, cotton, paper, diatomaceous earth, agar, gelatinous materials, polymeric materials, rock wool, glass wool, wood chips, bark, pumice and the like.

The composition and methods of the embodiments of the present invention may be useful on primed and unprimed seeds. Priming is a water-based process known in the art that is performed on seeds to increase uniformity of germination and emergence from a growing medium or soil, thus enhancing plant stand establishment. By incorporating the composition of the present invention into the priming process, or by incorporating at least one plant growth regulator into the priming process and applying at least one plant activator post-emergence, the benefits of optimum seed germination, optimum growth and development, synchronized time to flower, uniform flowering, uniformity in maturity of the crop, improved yields and improved quality of the harvested crop (fruit or other plant parts) are obtained. The time span between the emergence of the first and the last seedlings can be decreased more than with priming alone. As with priming, incorporation of the compositions and methods of the present invention into the priming process also increases the rate of emergence, so the plant stand establishes itself faster, ensuring maximum cartons of crop per acre at harvest. Wide ranges in seedling emergence decrease the amount of harvestable plants per acre, an undesirable situation for the commercial grower.

As used herein, a “container” refers to a structure having a defined space that can contain an amount of soil or other media in which a plant or a part of a plant, e.g., a seed, is grown. Typically, the plant or part of the plant is grown in the container, e.g., in a nursery, prior to transplantation to another site, such as another container or to an open soil bed.

An embodiment of the present invention provides methods and treatment combinations relating to reducing plant disease and/or pest/pathogen damage to a plant or protecting a plant against pest/pathogen damage, e.g., nematode disease. The methods therefore comprise a nematicide, such as an avermectin, e.g., abamectin, treatment in conjunction with biocontrol agent treatment, the combination of which results in improved plant growth or health in comparison to treatment with the individual agents. In typical embodiments, the biocontrol agent can inhibit nematodes or the diseases they cause.

The combination treatments of the invention can be used to control damage by any kind of pest, including nematodes, arthropods and the like. The treatments can be performed by treating a seed, seedling, or any part of a plant, with at least one nematicide, such as abamectin, and at least one biocontrol agent. Such a plant treatment can be performed by directly applying the at least one nematicide, such as abamectin, and/or at least one biocontrol agent to the plant, or by treating soil or other media in which the plant, or part of the plant, is sown.

In some embodiments, the at least one nematicide, such as but not limited to abamectin, and/or at least one biocontrol agent are used to control diseases caused by nematodes. Plant-parasitic nematodes that can be inhibited by using such a treatment regimen include root-knot, cyst, burrowing, dagger, lance, pin, reniform, lesion, ring, spiral, sting, stubby, stunt, stem and bulb, seed gall and foliar nematodes. In particular, nematodes of the following species can be managed using the combination treatments of the invention: Heterodera spp., e.g., H. schachtii, H. avenae, H. glycines, H. carotae, H. goettingiana, H. zeae and H. trifolii; Globodera spp., e.g., G. rostochiensis, G. pallida; Meloidogyne spp., e.g., M. incognita,M. javanica, M. hapla, M. arenaria, M. chitwoodi, M. graminis, M. mayaguensis, M. fallax, M. naasi; Radopholus spp., e.g., Radopholus similis, R. citrophilus; Pratylenchus spp., e.g., P. neglectans, P. scribneri, P. thornei, P. brachyurus, P. coffeae, P. zeae, and P. penetrans; Tylenchulus semipenetrans; Paratrichodorus minor, Longidorus spp., Helicotylenchus pseudorobustus, Hoplolaimus galeatus, H. columbus, H. tylenchiformis, Trichodorus proximus, Xiphinema index, X. americanum, Ditylenchus dipsaci, D. destructor, Nacobbus aberrans, Longidorus breviannulatus, L. africanus, Mesocriconema xenoplax, Aphelenchoides besseyi, A. fragariae, Zygotylenchus guevarai, Belonolaimus longicaudatus, B. gracilis, Anguina tritici, Rotylenchulus spp., Subanguina spp., Criconemella spp., Criconemoides spp., Dolichodorus spp., Hemicriconemoides spp., Hemicycliophora spp., Hirschmaniella spp., Hypsoperine spp., Macroposthonia spp., Melinius spp., Punctodera spp., Quinisulcius spp., Scutellonema spp., and Tylenchorhynchus spp.

Avermectins and derivatives of avermectins for use in the invention are known. Abamectin and abamectin seed treatment formulations for nematode control that are particularly useful in the invention are disclosed, e.g., in U.S. Pat. No. 6,875,727. Agrochemically compatible salts are, for example, acid addition salts of inorganic and organic acids, in particular of hydrochloric acid, hydrobromic acid, sulfuric acid, nitric acid, perchloric acid, phosphoric acid, formic acid, acetic acid, tri-fluoroacetic acid, oxalic acid, malonic acid, toluenesulfonic acid or benzoic acid. Examples of formulations of avermectin compounds that can be used in the method according to the invention, i.e., solutions, granules, dusts, sprayable powders, emulsion concentrates, coated granules and suspension concentrates, have been described, e.g., in EP-A-580 553.

Derivatives of avermectin or abamectin can be obtained via conventional chemical syntheses. For example, in some embodiments emamectin, which is 4″-De-oxy-4″-epi-N-methylamino avermectin B_{1b}/B₁ₐ known from U.S. Pat. No. 4,874,749, can be used. Agrochemically useful salts of emamectin are additionally described, e.g., in U.S. Pat. No. 5,288,710.

Abamectin for use in the invention can be applied to the soil or other growth media in which a seed or part of a plant to be propagated can be contained, or in other embodiments, can be formulated as a seed treatment pesticidal composition. Such abamectin-containing formulations are known in the art (see, e.g., U.S. Pat. No. 6,875,727).

The amount of a nematicide present on (or adhered to) the seed varies, for example, according to type of crop, and type of plant propagation material. However, the amount is such that the at least one nematicide is an effective amount to provide the desired enhanced action and can be determined by routine experimentation and field trials. In the event the nematicide is abamectin, the amount of active abamectin ingredient present in the seed coating is in the range of from 0.002 to 1.2 mg/seed, typically at least 0.1 mg/seed, often at least 0.2 mg/seed. Frequently, the abamectin is present at a level of 0.3 mg or more per seed.

Application of nematicide, such as abamectin, to a plant is described in greater detail below. One of ordinary skill in the art understands that the determination of the amount of nematicide, such as abamectin, depends on numerous factors, including the size of the plant material to be treated, for example, the size of the seed. One of ordinary skill can readily determine the amount of nematicide, such as abamectin, to employ based on the teachings in the art and known assays to validate the effects of applying the nematicide, e.g., assays described in the Examples section below

Any number of biocontrol agents can be used. Typical agents include bacteria, fungi, and other agents. Bacterial species that can be employed include members of a genus including Pasteuria, Pseudomonas, Corynebacterium, and Bacillus, as well as rhizobacteria, mycorrhizae, for example nematode-antagonistic mycorrhizae, and bacterial parasitic agents.

In some embodiments, the biocontrol agent that can be applied with the nematicide can be an anti-nematode biocontrol agent, e.g., an anti-nematode fungus, bacteria, or other agent. Nematode antagonistic bacteria include isolates of Agrobacterium sp, Bacillus sp., Myrothecium sp., and Pseudomonas sp. The modes of action of these bacteria are different, but include direct effects on egg hatching, mate and host finding, and nematode mobility as well as indirect effects, such as reduced root penetration.

Bacterial parasites can also be used as nematode antagonistic biocontrol agents. These include, e.g., Pasteuria species, e.g., P. penetrans, P. nishizawae, P. thornei, Candidatus Pasteuria usgae sp. nov., Myrothecium verrucaria, Candidatus Pasteuria sp. strain HG, and other species. These parasites can attach to the cuticle of nematodes

In some embodiments of the invention, nematode antagonistic fungi can be used. Such fungi include nematode-trapping fungi and parasitic fungi that are parasites of nematode juveniles, females, males and eggs. Nematode trapping fungi include species such as Arthrobotrys oligospora, A. conoides, A. musiformis, A. superba, A. thaumasia, A. dactyloides, A. haptotyla, Monoacrosporium psychrophilum, M. gephyropagum, M. elipsosporum, M. haptotylum, M. doedycoides, M. eudermatum, Duddingtonia flagrans, Dactylellina ellipsospora, Dactylella oxyspora, D. leptospora, D. rhopalota, Harposporium anguillulae, Meristacrum sp., Monacrosporium eudermatum, Nematoctonus leiosporus, and Stylopage sp.

Exemplary endoparasites include Drechmeria coniospora, Hirsutella rhossiliensis and Verticillium balanoides. These fungi produce spores that can attach to the nematode cuticle. Parasites of sedentary juvenile stages, females, males and/or eggs include Pochonia chlamydosporia, Paecilomyces lilacinus, Dactylella oviparasitica, Fusarium oxysporum, and Plectosphaerella cucumerina. Examples of fungi for use in the invention includes member of the following genera: Catenaria, Myzocytium, Haptoglossa, Meristacrum, Dactylella, Paecilomyces, Cephalosporium, Meria, Harposporium, Nematoctonus, Rhopalomyces, Verticillium, Pochonia, Saprolegnia, Cylindrocarpon, Nematophthora, Hirsutella, and Monoacrosporium.

Methods and combinations, especially compositions, of the invention can include additional pesticide components that exhibit either stimulatory or growth-promoting activity (e.g., nutrients, fertilizers, micronutrient donors, inoculants, antibiotics) towards the biological control agent(s), or inhibitory activity towards other pests, e.g., insecticides, acarcides, fungicides, other nematicides, or molluscides. Suitable additions of insecticidally, acaricidally, nematicidally, or molluscicidally active ingredients include, for example and not for limitation, the nematicides set forth above and representatives of the following classes of active ingredients: organophosphorus compounds, nitrophenols and derivatives, formamidines, triazine derivatives, nitroenamine derivatives, nitro- and cyanoguanidine derivatives, ureas, benzoylureas, carbamates, pyrethroids, chlorinated hydrocarbons, benzimidazoles, and Bacillus thuringiensis products. Especially preferred components in mixtures include cyanoimine, acetamiprid, nitromethylene nitenpyram, clothianidin, dimethoate, dinotefuran, fipronil, lufenuron, pyripfoxyfen, thiacloprid, fluxofenime; imidacloprid, thiamethoxam, beta cyfluthrin, fenoxycarb, lamda cyhalothrin, diafenthiuron, pymetrozine, diazinon, disulphoton; profenofos, furathiocarb, cyromazin, cypermethrin, tau-fluvalinate, tefluthrin, chlorantraniliprole or Bacillus thuringiensis products, very especially cyanoimine acetamiprid, nitromethylene nitenpyram, clothianidin, dinotefuran, dimethoate, lamda cyhalothrin, fipronil, thiacloprid, imidacloprid, thiamethoxam, beta cyfluthrin, chlorantraniliprole, and tefluthrin.

Suitable additions of fungicidally active ingredients include, for example and not for limitation, representatives of the following classes of active ingredients: strobilurins, triazoles, ortho-cyclopropyl-carboxanilide derivatives, phenylpyrroles, and systemic fungicides. Examples of suitable additions of fungicidally active ingredients include, but are not limited to, the following compounds: azoxystrobin; acibenzolor s-methyl, bitertanol; carboxin; Cu₂O; cymoxanil; cyproconazole; cyprodinil; dichlofluamid; difenoconazole; diniconazole; epoxiconazole; fenpiclonil; fludioxonil; fluoxastrobin, fluquiconazole; flusilazole; flutriafol; furalaxyl; guazatin; hexaconazole; hymexazol; imazalil; imibenconazole; ipconazole; kresoxim-methyl; mancozeb; metalaxyl; R metalaxyl; metconazole; myclobutanil, oxadixyl, pefurazoate; penconazole; pencycuron; picoxystrobin; prochloraz; propiconazole; pyroquilone; SSF-109; spiroxamin; tebuconazole; tefluthrin; thiabendazole; thiram, tolifluamide; triazoxide; triadimefon; triadimenol; trifloxystrobin, triflumizole; triticonazole and uniconazole. Particularly preferred fungicidally active agents include azoxystrobin, acibenzolor s-methyl, difenoconazole, fludioxonil, metalaxyl, R-metalaxyl, myclobutanil, thiabendazole, a compound of formula A, a compound of formula B, and trifloxystrobin.

Suitable additional pesticides for use in the invention can be selected such that the biocontrol agent is resistant to the pesticide agent. For example, when a biocontrol fungus is employed, additional fungicides that may be included in the treatments can be selected for uses that do not inhibit the growth of the biocontrol fungus.

In some embodiments in which the nematicide such as abamectin and/or biocontrol agent is administered by treating the soil or other media, the nematicide and/or biocontrol agent is applied to the site where the plant or part of the plant has been, or will be sown. For example, the nematicide or biocontrol agent can be applied prior to sowing into the seed furrow or to an area around the site of planting or sowing the propagation material, such that the nematicide or biological control agent can effectively inhibit nematode hatch, growth, host or mate finding and/or protect plant tissues against nematode feeding. The agents can also be administered during planting or following planting at a time that effectively controls nematode growth.

As noted, in some embodiments, a plant or part of a plant can be treated with the nematicide and/or biocontrol agents. Treatment can be performed using a variety of known methods, e.g., by spraying, atomizing, dusting or scattering the compositions over the propagation material or brushing or pouring or otherwise contacting the compositions over the propagation material or, in the event of seed, by coating, encapsulating, or otherwise treating the seed.

For application of the pesticidal composition as a seed treatment, the at least one nematicide, such as an avermectin, with or without additional pesticidal agents, is added to the seed, typically prior to sowing or while planting, and the active substances are distributed over the seed. Particular embodiments of such a seed treatment comprise, for example, immersing the seed in a liquid composition, coating the seed with a solid composition or by achieving penetration of the active ingredient into the seed, e.g., by adding the composition to water used for pre-soaking seeds. The rates of application of the pesticidal composition can vary, for example, according to type of use, type of crop, the specific active ingredients in the pesticidal composition, and type of plant propagation material, but is such that the active ingredients in the combination are an effective amount to provide the desired enhanced action and can be determined by routine experimental trials. Typical application rates of the compositions seeds can be, for example, between 0.1 and 1000 g of active ingredient per 100 kg of seed; in particular, between 1 and 600 g/100 kg of seed; preferable between 1 and 400 g/100 kg of seed; and especially 1 to 200 g/100 kg of seed.

In other embodiments, the plant seed can be treated with the nematicidal agent, preferably with an avermectin-containing, e.g., abamectin-containing, pesticidal agent, by applying the nematicidal agent to the soil or other media in which the seed is planted, e.g., the planting media in a container for a nursery plant. This can be administered in any known method, for example, by spraying, scattering, pouring and the like. The application rates may vary within wide ranges and depend on the soil constitution, the type of application (foliar application; application in the seed furrow), the plant, the pest/pathogen to be controlled, the climatic circumstances prevailing in each case, and other factors determined by the type of application, timing of application and target crop. With abamectin, the application rates per hectare are generally 1 to 2000 g abamectin per hectare; in particular 10 to 1000 g/ha; preferably 10 to 500 g/ha; especially preferably 10 to 200 g/ha. In some embodiments, 1 to 100 g/ha, e.g., 1 to 50 g/ha, or 1 to 25 g/ha may be used.

The methods of the invention additionally can comprise applying at least one or more biocontrol agents to plants, plant seeds, soil or other media surrounding plants under conditions where the biocontrol agent reduces susceptibility to pests or pathogens, e.g., plant-parasitic nematodes. Application of at least one or more biocontrol agents in combination with a nematicide, such as an avermectin (e.g., abamectin), also provides a method of enhancing plant growth and improving plant vigor.

Application of at least one biocontrol agent directly to a plant can be performed using methods in which all or a part of the plant is directly treated. Typically, plant seed is treated, but other parts of the plant, such as propagating material, may also be directly treated. Suitable application methods include high or low pressure spraying, drenching, and injection. In other embodiments, the biocontrol agent can be added to seeds (or the soil or other planting media) as the seeds are being planted. It is understood that the plants may be further treated with other nematicides, e.g., abamectin, aldicarb, and the like, and at least one biocontrol agent after seeds have been planted. Thus, the invention includes embodiments in which plants may be treated with one or more applications of the at least one biocontrol agent and at least one nematicide to provide enhanced pest resistance to plants and/or to enhance plant growth.

The biocontrol agents can be applied to plants or plant propagation material, such as seeds, in accordance with the present invention alone or in a mixture with other compounds, e.g., a pesticidal composition comprising abamectin. Alternatively, the at least one biocontrol agent can be applied separately to plants and other compounds, e.g., the abamectin-containing composition, applied at different times.

The at least one biocontrol agent can be applied directly to the plant propagation material, such as seed, prior to sowing it in the field. In its simplest form, this can be done by spraying or dipping the plant propagation material, such as seed, with a liquid culture containing an anti-nematode fungal strain and/or bacterial strain and/or other biocontrol agent.

A composition suitable for treating plants or plant propagation material, such as seeds, in accordance with the present invention often contains a biocontrol agent in a carrier. Thus, the at least one biocontrol agent can be applied to plant propagation material, such as seeds, with other conventional seed formulations and treatments and treatment materials. Suitable additives include buffering agents, wetting agents, coating agents, polysaccharides, and abrading agents. Exemplary carriers include water, aqueous solutions, slurries, solids and dry powders (e.g., peat, wheat, bran, vermiculite, clay, pasteurized soil, many forms of calcium carbonate, dolomite, various grades of gypsum, bentonite and other clay minerals, rock phosphates and other phosphorous compounds, titanium dioxide, humus, talc, alginate and activated charcoal. Any agriculturally suitable carrier known to one skilled in the art would be acceptable and is contemplated for use in the present invention.

In some embodiments, e.g., when using bacterial or fungal biocontrol agents, an adhesive can be included to hold the bacteria-containing propagules to the seed. Such adhesives are known in the art. Exemplary agents include glues and gums, e.g., of plant or microbial origin, gelatin, sugars, and the like.

One of ordinary skill in the art understands that agents that are included as a carrier are selected to not adversely affect the growth of the biocontrol agent or plant.

In an alternative to directly treating a seed before planting, a biocontrol agent can also be introduced into the soil or other media into which the seed is to be planted. Typically, a carrier is also used in this embodiment. The carrier can be solid or liquid, as noted above.

In some embodiments a popular method is to employ peat suspended in water as a carrier of the biocontrol agent, and spray this mixture into the soil or planting media and/or over the seed as it is planted. Other examples of a solid agricultural inoculum that can be used in applying the biocontrol agent to the soil (or seed as it is planted) are granules comprised of calcium sulfate hemihydrate and carboxymethylcellulose sprayed with a bacterial broth or a fungi-containing broth or another similar biocontrol agent broth. Peat or soil inoculated with the at least one biocontrol agent are also examples of materials that can be used in applying the at least one biocontrol agent to the soil or plant propagation material as it is planted.

In some embodiments, the at least one biocontrol agent may be applied to a young plant, e.g., can be added to the soil or other growth media in which a seedling is growing following planting.

The combination treatment of at least one nematicide, such as abamectin, and at least one biocontrol agent can be applied at a density sufficient to cover the area where nematode growth is expected to be observed. For example, a formulation containing at least one biocontrol agent can be applied to soil in amounts of about 0.1 gallons per acre to about 300 gallons per acre, wherein the formulation is at a concentration of about 10⁴ to about 10¹² spores or cfu per ml as a liquid formulation, or at a concentration of about 10⁴ to about 10¹² spores or cfu per gram as a solid formulation.

The at least one nematicide-containing composition and at least one biocontrol agent can be administered in a “pesticidally effective” amount. A pesticidally effective amount is considered to be an amount at which the combination treatment enhances pesticide efficacy and/or duration and/or improves plant growth. It is understood that an effective amount of agent may not reduce the numbers of pests/pathogens, e.g., nematode eggs, per se, but is effective in decreasing damage to plants as a result of a pest/pathogen such as a nematode. Accordingly, the efficacy of a treatment can be assessed via any direct or indirect endpoints. For example, a pesticidally effective amount may reduce pest damage to seeds, roots, shoots, or foliage of plants that are treated compared to those that are untreated.

In preferred embodiments, the combination treatment of at least one nematicide and at least one biocontrol agent can, with or without additional pesticides, use amounts of the two agents that are sufficient to control nematode-caused plant disease. “Controlling nematode-caused plant disease” refers to the ability of a combination treatment of the invention to influence nematode population density and/or their activity to a degree sufficient to reduce or prevent nematodes form detrimentally affecting the growth of the surrounding plants. “Controlling” nematode-caused plant disease does not necessarily require the eradication of all of the nematodes in an area. Nematode population density and/or activity can be effectively inhibited if the plant exhibits symptoms of nematode-related disease that are reduced in comparison to those of a control plant not treated with the combination.

Plants that can be treated in accordance with the embodiments of the invention include both monocotyledonous and dicotyledonous plant species including cereals such as barley, rye, sorghum, tritcale, oats, rice, wheat, soybean, corn,; beets (for example sugar beet and fodder beet); cucurbits including cucumber, muskmelon, canteloupe, squash and watermelon; cole crops including broccoli, cabbage, cauliflower, bok choi, and other leafy greens, other vegetables including tomato, pepper, lettuce, beans, pea, onion, garlic and peanut; oil crops including canola, peanut, sunflower, rape, and soybean; solanaceous plants including tobacco; tuber and root crops including potato, yam, radish, beets, carrots and sweet potatoes; fruits including strawberry; fiber crops including cotton, flax and hemp; other plants including coffee, bedding plants, perennials, woody ornamentals, turf and cut flowers including carnation and roses; sugar cane; containerized tree crops; evergreen trees including fir and pine; deciduous trees including maple and oak; and fruit and nut trees including cherry, apple, pear, almond, peach, walnut and citrus. In general any plant that is susceptible to plant disease and/or pest damage (e.g., insect or nematode damage) and responds to the combination of the invention may be treated in accordance with the invention.

In some embodiments, the nematicide, preferably avermectin, such as abamectin, containing composition and the at least one biocontrol agent can be applied to plant propagation material, such as seeds or other plant material, that are to be transplanted and/or that are to be grown in a nursery. Such plants are typically grown in containers. Thus, in some embodiments, the at least one biological control agent can conveniently be added to the soil or other planting media in the container. In an embodiment, a pesticidal composition comprising abamectin can be applied directly to a plant or part of the plant, such as the seed. Alternatively, the abamectin-containing composition may be added to the soil or other planting media in the container in which the plant is to be grown. In some embodiments, the plants may receive multiple treatments with abamectin and/or the at least one biocontrol agent. Further, the plants may be treated with additional agents, e.g., a second biological control agent or another nematicide, pesticides, fungicides, etc.

Treatment of nursery plants, e.g., seeds or seedlings, with a combination treatment of the invention results in improved growth of the plants due to decreased damage by pests or pathogens, such as nematodes. After initial growth in a container, the plant can be transferred to another container or open bed. In some embodiments, the plants may be subjected to further treatments with abamectin and/or the biocontrol agent following or during transplantation.

The invention thus also relates to compositions comprising a container, soil or other planting media, a plant, abamectin, and at least one biological control agent. Such a composition is typically a container that has soil or other planting media into which at least one biological control agent has been introduced and one or more abamectin-treated seeds have been planted. The at least one biological control agent in some embodiments may be introduced by treating seeds with the agent.

The present invention therefore envisages treating a plant propagation material with a pesticide composition comprising one or more nematicide and applying one or more biocontrol agents to the locus of the plant propagation material; treating a plant propagation material with a pesticide combination composition; treating a plant propagation material with one or more biocontrol agents and applying a pesticide composition comprising one or more nematicides to the locus of the plant propagation material; or applying a pesticide combination composition to the locus of the plant propagation material.

The following examples are provided by way of illustration only and not by way of limitation. Those of ordinary skill in the art will readily recognize a variety of non-critical parameters that could be changed or modified to yield essentially similar results.

### EXAMPLES

These examples evaluate abamectin seed treatment in combination with nematode-destroying fungi in trials with cucumber and tomato.

In Examples 1-3, a strain of the nematode-destroying fungus Pochonia chlamydosporia was used. This fungal species, formerly called Verticillium chlamydosporium, has been extensively researched for biological control of endoparasitic nematodes (see, e.g., Kerry and Bourne, A manual for research on Verticillium chlamydosporium, a potential biological control agent for root-knot nematodes, IOBC/OILB, Druckform GmbH, Darmstadt, Germany, 2002).

### Example 1

### Cucumber Green House Trials

Pots (10-cm diameter) were filled with 250 g (dry weight) steam-pasteurized river bottom sand. Ten treatments with 6 replications were prepared (Table 1). The fungal antagonist Pochonia chlamydosporia was grown on autoclaved moist millet seed for three weeks at 22° C. Colonized millet was dried in a laminar flow hood, and stored aseptically at 4° C. until use. For soil inoculation, P. chlamydosporia-colonized millet was thoroughly mixed with the sand. Population density of the fungus was approximately 2000 chlamydospores/cc soil for rate 1 and 4000 chlamydospores/cc soil for rate 2.

TABLE 1
Greenhouse trial treatment list

| Treatment no. | Rk-nematodes | P. chlamydosporium | abamectin |
|---|---|---|---|
| 1 | no | no | no |
| 2 | yes | no | no |
| 3 | yes | rate 1 | no |
| 4 | yes | rate 2 | no |
| 5 | yes | no | 0.1 mg/seed |
| 6 | yes | rate 1 | 0.1 mg/seed |
| 7 | yes | rate 2 | 0.1 mg/seed |
| 8 | yes | no | 0.3 mg/seed |
| 9 | yes | rate 1 | 0.3 mg/seed |
| 10 | yes | rate 2 | 0.3 mg/seed |

The nematode inoculum was raised during the previous 3 months on tomato plants (Lycopersicum esculentum cv. Tropic) in the greenhouse. Nematode eggs were obtained by standard bleach/sieving extraction. With the exception of the first treatment, each pot was infested with ca. 30000 eggs of M. incognita. This is a typical infestation level for nematicide tests resulting in high disease pressure (expected gall rating for non-treated control at 8 weeks was approximately 7 on a scale of 0-10 (Zeck, Pflanzenschutz-Nachrichten, Bayer AG, 24:141-144, 1971). Cucumber seeds (Cucumis sativus L. cv. Straight Eight, Burpee Seed Co.) were coated with either0.1 mg or 0.3 mg abamectin/seed or received no further treatment. Each pot received slow-release fertilizer (Osmocote Vegetable and Bedding Plant Food, 14-14-14, The Scotts Company) recommended for tomato production. The pots were arranged in a randomized complete block design in a greenhouse at ca. 24°±3° C. and ambient lighting. Irrigation was applied daily as needed. Three and eight weeks after seeding plant height or main vine length was determined. Eight weeks after seeding the trial was terminated and the plant tops were cut off. They were placed in a drying oven overnight and their weight was determined. The roots were placed in eryoglaucin solution overnight and the stained egg masses of the root-knot nematodes were counted. Root galling was rated on a scale of 0-10 (0=no galling). The trial was repeated once.

### Results

### Cucumber Seed Coating Trial 1

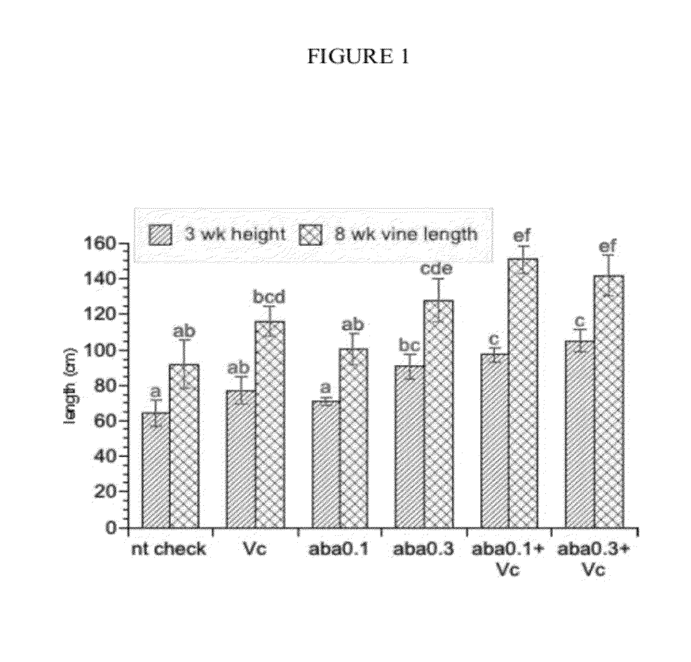

The trial was of high quality. No other disease incidence was noted in the trial. Early growth differences among the treatments were observed and documented (Table 2). The low rate of abamectin did not show a benefit for the crop as neither plant growth was improved nor root galling was significantly reduced (Table 2). Similarly, the low rate of Pochonia did not have any significant influence on plant growth and galling. The high rate of Pochonia by itself was not much better in terms of growth promotion or gall reduction. In contrast, the protection from nematode-attack by the 0.3 mg/seed rate of abamectin caused significant increases in early plant growth as well as in plant dry weight and main vine length at termination of the trial compared to the non-treated control plants. The combination of either rate of abamectin with the high rate of Pochonia outperformed all other treatments in nearly all parameters and was, in terms of plant performance, not significantly different from the nematode-free control (Table 2). An analysis of the combination treatment results is shown in FIG. 1. The nematode population was expressed in terms of egg masses. The non-treated control had the most egg masses and all treatments resulted in significant reductions. However, due to the large variability in egg mass number, no significant differences were found among the treatments (Table 2).

TABLE 2
Plant growth and nematode population determinations during cucumber trial 1

| | plant | plant dry | main vine | | |
|---|---|---|---|---|---|
| | height (mm) @ | weight (g) @ | length (cm) @ | number egg | root galling @ |
| Treatments | 3 weeks^{a} | 8 weeks^{a} | 8 weeks^{a} | masses/root^{a} | 8 weeks^{a} |
| 1. nt, n-inf. control | 101.0 ± 6.1 c | 7.4 ± 0.2 d | 164.0 ± 6.7 f | 0.0 ± 0.0 | 0.0 ± 0.0 a |
| 2. nt, rkn check | 64.2 ± 7.2 a | 4.1 ± 0.8 a | 92.0 ± 13.6 ab | 139.3 ± 46.2 b | 6.0 ± 0.4 cde |
| 3. nt + Pc1, rkn | 64.3 ± 3.7 a | 3.8 ± 0.7 a | 89.0 ± 8.4 a | 85.4 ± 12.1 ab | 6.8 ± 0.6 e |
| 4. nt + Pc2, rkn | 77.2 ± 8.0 ab | 4.9 ± 0.5 ab | 116.2 ± 8.4 bcd | 93.2 ± 14.4 ab | 5.6 ± 0.4 cde |
| 5. aba0.1, rkn | 70.8 ± 2.3 a | 4.2 ± 0.4 a | 100.7 ± 8.8 ab | 108.2 ± 14.0 ab | 6.3 ± 0.6 de |
| 6. aba0.1 + Pc1, rkn | 67.8 ± 5.4 a | 4.3 ± 0.4 a | 108.2 ± 11.0 abc | 76.7 ± 16.2 a | 6.5 ± 0.6 e |
| 7. aba0.1 + Pc2, rkn | 97.3 ± 4.3 c | 6.9 ± 0.2 cd | 150.8 ± 7.7 ef | 106.2 ± 20.9 ab | 4.5 ± 0.3 bc |
| 8. aba0.3, rkn | 90.8 ± 7.1 bc | 5.7 ± 0.4 bc | 127.8 ± 11.9 cde | 85.8 ± 12.8 ab | 5.8 ± 0.4 cde |
| 9. aba0.3 + Pc1, rkn | 94.0 ± 6.0 c | 6.0 ± 0.3 bc | 138.7 ± 9.0 de | 86.5 ± 14.4 ab | 5.0 ± 0.5 bcd |
| 10. aba0.3 + Pc2, rkn | 105.2 ± 6.1 c | 6.3 ± 0.3 cd | 141.7 ± 11.2 ef | 74.2 ± 17.5 a | 4.0 ± 0.7 b |

nt = no seed treatment; n-inf. = no rkn (root-knot nematodes, Meloidogyne incognita race 1); Pc 1 = Pochonia chlamydosporia rate 1 (2000 chlamydospores/g soil); Pc 2 = P. chlamydosporia rate 2 (4000 chlamydospores/g soil); aba = abamectin seed coating at 0.1 or 0.3 mg/seed).
^{a}Means with standard error (P = 0.05). Identical letters in the same column indicate results do not significantly differ.

### Cucumber Seed Coating Trial 2

The quality of the second trial was good. No other disease incidence was noted. The results were similar to the first trial. Early protection against the root-knot nematodes resulted in obvious and significant plant growth differences compared to the untreated control (Table 3). Plant dry weight and vine length were increased by all treatments compared to the untreated control (Table 3). As in the first trial, the number of egg masses did not differ greatly among the treatments. This is mainly due to the stunted plant growth and poor root system in the untreated that did not offer sufficient feeding sites for the nematodes (treatment 2). Consequently, a nematode-protected and therefore larger root system may have at the end of the season a larger nematode population than that the control. The combination of the high rate of abamectin and the high rate of P. chlamydosporia again resulted in the lowest gall rating (Table 3).

TABLE 3
Plant growth and nematode population determinations during cucumber trial 2

| | | | | number egg | |
|---|---|---|---|---|---|
| | plant | plant dry | main vine | masses/root | root galling @ |
| | height (mm) @ | weight (g) @ | length (cm) @ | system @ | 8 weeks |
| Treatments | 3 weeks | 8 weeks | 8 weeks | 8 weeks | (scale 0-10) |
| 1. nt, n-inf. control | 108.5 ± 5.5 de | 7.5 ± 0.2 d | 174.8 ± 2.3 d | 0.0 ± 0.0 | 0.0 ± 0.0 a |
| 2. nt, rkn check | 58.0 ± 8.2 a | 4.1 ± 0.4 a | 104.0 ± 9.6 a | 44 ± 8.3 ab | 8.2 ± 0.5 d |
| 3. nt + Pc1, rkn | 93.8 ± 3.9 bc | 5.1 ± 0.4 b | 117.7 ± 6.3 ab | 80.8 ± 18.0 b | 5.8 ± 0.2 bc |
| 4. nt + Pc2, rkn | 82.5 ± 6.6 b | 5.6 ± 0.4 bc | 133.2 ± 9.6 bc | 59.2 ± 14.3 ab | 6.7 ± 0.5 c |
| 5. aba0.1, rkn | 96.2 ± 6.4 bcd | 6.4 ± 0.5 c | 146.2 ± 9.8 c | 80.8 ± 27.8 b | 5.3 ± 0.2 abc |
| 6. aba0.1 + Pc1, rkn | 102.5 ± 5.3 cde | 5.5 ± 0.3 bc | 131.7 ± 4.3 bc | 52.2 ± 9.5 ab | 5.0 ± 3.0 ab |
| 7. aba0.1 + Pc2, rkn | 99.0 ± 6.0 cde | 6.0 ± 0.4 bc | 149.5 ± 11.0 c | 35.3 ± 10.5 a | 5.5 ± 0.3 bc |
| 8. aba0.3, rkn | 105.8 ± 5.3 cde | 6.0 ± 0.3 bc | 131.3 ± 6.2 bc | 36.7 ± 4.0 a | 5.0 ± 0.4 ab |
| 9. aba0.3 + Pc1, rkn | 108.0 ± 3.0 cde | 6.1 ± 0.4 c | 146.7 ± 7.3 c | 34.5 ± 9.3 a | 5.0 ± 0.7 ab |
| 10. aba0.3 + Pc2, rkn | 111.3 ± 4.7 e | 6.4 ± 0.4 c | 144.0 ± 7.3 c | 44.8 ± 10.7 ab | 4.0 ± 0.8 a |

nt = no seed treatment; n-inf. = no rkn (root-knot nematodes, Meloidogyne incognita race 1); Pc 1 = Pochonia chlamydosporia rate 1 (2000 chlamydospores/g soil); Pc 2 = P. chlamydosporia rate 2 (4000 chlamydospores/g soil); aba = abamectin seed coating at 0.1 or 0.3 mg/seed).
^{a}Means with standard error (P = 0.05). Identical letters in the same column indicate results do not significantly differ.

### Example 2

### Tomato Green House Trials

The greenhouse trials were conducted in pulp pots (10-cm diameter) filled with steam-pasteurized sand (250 cm³). The biological control organism (BCO) P. chlamydosporia was grown as described above. P. chlamydosporium-inoculated millet seed were washed (1:2 weight/volume millet seed and sterile distilled water, 2 min shaking in electric blender) and passed through a 100-mesh sieve to remove the millet from the fungal chlamydospores. These served as the inoculum and were counted with a counting chamber (Fuchs-Rosenthal). The chlamydospores were thoroughly mixed with the sand. Population density of fungus was approximately 2000 chlamydospores/g soil for rate 1 and 4000 chlamydospores/g soil for rate 2 (Table 1). Tomato seeds (Lycopersicum esculentum cv. Tiny Tim) were coated with either 0.1 mg or 0.3 mg abamectin/seed or received no further treatment (Table 1). The tomato seeds were sown into seeding trays with commercial seedling substrate and after 2 weeks the plants were transplanted into 10-cm pulp pots. With the exception of the first treatment, each pot was infested with ca. 30000 eggs of M. incognita. Egg hatch rate was approximately 10% on Baerman funnels at 26° C. for 5 days. Each pot received slow release fertilizer (Osmocote Vegetable and Bedding Plant food, 14-14-14, The Scotts Company). Pots were arranged in a randomized complete block design with 6 replications per treatment and incubated in greenhouse at ca. 24±3C and ambient lighting. Plants were watered daily as needed. Plant height was determined and the shoots were cut off at the end of the trial. Shoots be placed in a drying oven at 69° C. for 72 h and the weight of each plant was determined. The extent of root galling was assessed on a scale from 0-10 (Zeck, 1971, supra).

Nematode populations were determined by counting egg masses (=number of fecundate females), eggs and second-stage juvenile (J2). The roots were placed in eryoglaucin solution overnight to stain egg masses of the root-knot nematodes which enabled their enumeration (Omwega et al., 1988). The eggs were released from egg masses through a modified bleach/sieving extraction technique (Hussey and Barker, 1973). Each week, ripe (red) tomato fruits were picked off, and the number and weight were recorded. Picking was continued until fruit production seized. The trial was repeated once. All data were subject to analysis of variation with SuperANOVA (Abacus Concepts, 1989, Berkeley, Calif.). If appropriate, Fisher's Protected Least Significant Difference (LSD) was used to separate means at P=0.05.

### Results

Trial qualities were both excellent and the results were similar. Thus, the data were therefore combined for analysis. All treatments increased plant height and dry weight compared to the non-treated check (Table 4). Generally, the combination treatments resulted in the tallest plants and the ones with the highest dry weight. Despite the very severe root-knot nematode infestation, the high abamectin seed coating rate combined with the high BCO rate resulted in dry weights similar to the non-infested control. Root galling was reduced by abamectin to approximately two rating classes below the check. This efficacy is typical for the abamectin seed coating. While the combination with the BCO improved only slightly the efficacy of the low abamectin rate, the galling was dramatically reduced in both combination treatments with either rate of P. chlamydosporia.

TABLE 4
Tomato growth comparisons at tomato greenhouse trial
termination (data of two trials combined).

| | Plant | Plant dry | |
|---|---|---|---|
| No. of Treatment | height(cm)^{a} | weight(g)^{a} | Root galling^{a} |
| 1. nt, n-inf. control | 28.50 ± 1.61cd | 8.16 ± 0.18e | 0.00 ± 0.00a |
| 2. nt, rkn check | 19.67 ± 1.23a | 3.18 ± 0.38a | 8.33 ± 0.21g |
| 3. nt + Pc 1, rkn | 26.00 ± 0.86bc | 5.23 ± 0.42b | 7.33 ± 0.33fg |
| 4. nt + Pc 2, rkn | 24.00 ± 1.29b | 4.89 ± 0.30b | 6.17 ± 0.40de |
| 5. aba0.1, rkn | 27.33 ± 0.72cd | 5.24 ± 0.35b | 6.67 ± 0.21ef |
| 6. aba0.1 + Pc 1, rkn | 27.83 ± 0.65cd | 6.78 ± 0.48cd | 5.67 ± 0.49cde |
| 7. aba0.1 + Pc 2, rkn | 29.67 ± 0.96d | 7.87 ± 0.55e | 5.50 ± 0.50cd |
| 8. aba0.3, rkn | 26.17 ± 2.14bc | 6.31 ± 0.21c | 5.00 ± 0.52c |
| 9. aba0.3 + Pc 1, rkn | 27.33 ± 0.96cd | 7.74 ± 0.18de | 2.17 ± 0.17b |
| 10. aba0.3 + Pc 2, rkn | 29.50 ± 1.29d | 8.03 ± 0.36e | 3.00 ± 0.63b |

nt = no seed treatment; n-inf. = no rka (root-knot nematodes, Meloidogyne incognita race 1); Pc 1 = Pochonia chlamydosporia rate 1 (2000 chlamydospores/g soil); Pc 2 = P. chlamydosporia rate 2 (4000 chlamydospores/g soil); aba = abamectin seed coating at 0.1 or 0.3 mg/seed).
^{a}Means with standard error (P = 0.05). Identical letters in the same column indicate results do not significantly differ.

The number of reproductive females, indicated by the number of eggs, did not substantially differ, indicating that the BCO did not parasitize the developing or adult nematodes (Table 5). The number of eggs varied considerably and only the treatments with the high abamectin rate had lower egg numbers than the check. Similar results were obtained by extraction of J2 from soil.

TABLE 5
Root-knot nematode populations at tomato greenhouse
trial termination (data of two trials combined).

| treatment | egg masses/root^{a} | egg count/root^{a} | J2/50 ml soil^{a} |
|---|---|---|---|
| 1. nt, n-inf. control | 0.0 ± 0.0a | 0.0 ± 0.0a | 0.0 ± 0.0a |
| 2. nt, rkn check | 454.2 ± 85.5c | 35913 ± 92328d | 56.5 ± 33.3bc |
| 3. nt + Pc 1, rkn | 377.5 ± 53.5c | 276800 ± 27844cd | 84.5 ± 31.7c |
| 4. nt + Pc 2, rkn | 444.2 ± 80.2c | 261266 ± 32880cd | 22.8 ± 13.1ab |
| 5. aba0.1, rkn | 454.2 ± 62.7c | 346133 ± 39003d | 20.3 ± 4.3ab |
| 6. aba0.1 + Pc 1, rkn | 418.3 ± 69.3c | 293866 ± 33768cd | 54.3 ± 33.2bc |
| 7. aba0.1 + Pc 2, rkn | 475.8 ± 89.1c | 247466 ± 35400cd | 16.3 ± 7.0ab |
| 8. aba0.3, rkn | 381.7 ± 120.3c | 205867 ± 66056bc | 7.2 ± 5.4ab |
| 9. aba0.3 + Pc 1, rkn | 147.5 ± 24.0ab | 100800 ± 23468ab | 3.0 ± 2.9ab |
| 10. aba0.3 + Pc 2, rkn | 315.0 ± 97.9bc | 193600 ± 39476bc | 3.5 ± 1.9ab |

nt = no seed treatment; n-inf. = no rkn (root-knot nematodes, Meloidogyne incognita race 1); Pc 1 = Pochonia chlamydosporia rate 1 (2000 chlamydospores/g soil); Pc 2 = P. chlamydosporia rate 2 (4000 chlamydospores/g soil); aba = abamectin seed coating at 0.1 or 0.3 mg/seed).
^{a}Means with standard error (P = 0.05). Identical letters in the same column indicate results do not significantly differ.

All treatments increased the number of fruits per plant, the total fruit weight as well as the average fruit weight compared to the non-treated check (Table 6). The combination of the high rates of abamectin and P. chlamydosporium had the most fruits and highest total fruit weight.

TABLE 6
Tomato yield in greenhouse trial (data of two trials combined).

| | number of fruits/ | total fruit weight/ | |
|---|---|---|---|
| treatment | plant^{a} | plant (g)^{a} | fruit weight (g)^{a} |
| 1. nt, n-inf. control | 58.2 ± 3.9f | 313.2 ± 23.9e | 5.42 ± 0.38bc |
| 2. nt, rkn check | 9.5 ± 2.3a | 42.1 ± 10.7a | 3.72 ± 0.80a |
| 3. nt + Pc 1, rkn | 21.5 ± 5.7ab | 106.6 ± 27.3ab | 5.05 ± 0.29bc |
| 4. nt + Pc 2, rkn | 28.0 ± 3.9bc | 142.6 ± 16.8b | 5.16 ± 0.13bc |
| 5. aba0.1, rkn | 31.0 ± 4.6bcd | 160.3 ± 22.8bc | 5.19 ± 0.18bc |
| 6. aba0.1 + Pc 1, rkn | 34.2 ± 5.9cd | 160.7 ± 28.0bc | 4.65 ± 0.17ab |
| 7. aba0.1 + Pc 2, rkn | 41.8 ± 3.7de | 217.7 ± 23.2cd | 5.24 ± 0.38bc |
| 8. aba0.3, rkn | 40.5 ± 1.5cde | 243.3 ± 11.8d | 6.00 ± 0.14c |
| 9. aba0.3 + Pc 1, rkn | 41.2 ± 3.8de | 218.9 ± 33.7cd | 5.19 ± 0.39bc |
| 10. aba0.3 + Pc 2, rkn | 47.8 ± 6.6ef | 259.6 ± 30.2de | 5.59 ± 0.36bc |

nt = no seed treatment; n-inf. = no rkn (root-knot nematodes, Meloidogyne incognita race 1); Pc 1 = Pochonia chlamydosporia rate 1 (2000 chlamydospores/g soil); Pc 2 = P. chlamydosporia rate 2 (4000 chlamydospores/g soil); aba = abamectin seed coating at 0.1 or 0.3 mg/seed).
^{a}Means with standard error (P = 0.05). Identical letters in the same column indicate results do not significantly differ.

### Example 3

### Tomato Miniplot Field Trial

Nine miniplots (3 m diameter, 12 cm deep) were each filled with approximately 350000-cm³ field soil (sandy loam, pH 7.2) obtained from an adjacent field with no significant infestation of plant-parasitic nematodes. Tomato seedlings (Lycopersicum esculentum cv. Tiny Tim) were raised from abamectin-treated seed (0.3 mg a.i./seed) or from Apron/Maxim-treated seed. They were seeded in seedling trays with commercial transplant substrate (Sunshine mix). The substrate was either non-amended or amended with P. chlamydosporia (4000 chlamydospores/cm³ substrate). After 3 weeks in a greenhouse, the seedlings were transplanted into the 9 miniplots. Each plot was a randomized block with 4 treatments and three plants per treatment. Each planting area was infested by distributing 10,000 eggs of M. incognita race 1 into three 5 cm deep holes approximately 5 cm from each transplant. The plots were irrigated via low-pressure irrigation and fertilized according to local standard. After approximately 10 weeks the plants set fruit and were harvested three times during the following 3 weeks. Number of fruits and weight were taken. All data were subject to ANOVA and mean separation with Fisher's LSD (P=0.05).

### Results

The trial quality was excellent. Both the nematicidal seed coating and the BCO increased the yield significantly (Table 7). Both the average number of fruits per plant and the average total fruit weight increased in response to the treatments. In contrast to earlier trials, the BOC did not differ from the chemical treatment in terms of yield response. However, both single applications were outperformed by the combined treatment of P. chlamydosporia and abamectin. In contrast to the greenhouse trials, egg population at harvest was the highest in the combined treatment. This may be an indication for the role of other microorganisms that in natural field soil frequently enhance the destruction of roots parasitized by root-knot nematodes. Protected roots typically have the largest and healthiest root system thus providing abundantly feeding sites for the nematodes.

TABLE 7
Tomato yield in miniplot field trial

| | number | total fruit | root galling | M. incognita |
|---|---|---|---|---|
| Treatments | fruits/plant^{a} | weight/plant^{a} | at harvest^{a} | eggs/plant^{a} |
| Non-treated control | 55.8 ± 5.5 a | 214.7 ± 23.9 a | 8.1 ± 0.3 a | 134,500 ± 30,300 a |
| P. chlamydosporia | 70.7 ± 4.1 b | 271.5 ± 17.4 b | 5.8 ± 0.5 b | 216,600 ± 34,700 ab |
| abamectin 0.3 mg/seed | 69.0 ± 3.2 b | 290.6 ± 12.6 b | 4.5 ± 0.2 c | 171,400 ± 37,100 a |
| P. chlamydosporia + | 81.4 ± 2.9 c | 321.8 ± 20.6 c | 4.7 ± 0.3 c | 306,300 ± 46,000 b |
| abamectin 0.3 mg/seed | | | | |

^{a}Means with standard error (P = 0.05). Identical letters in the same column indicate results do not significantly differ.

The results presented in these examples demonstrated that the combination of abamectin seed coating with a nematode-destroying fungus P. chlamydosporium is a successful novel strategy to utilize the strength of both systems while helping to overcome their individual shortcomings.

### Example 4

### Root-Knot Nematode Trials

In this project we evaluated the potential benefits of combinations of abamectin seed coating with soil applications of Pasteuria penetrans on the efficacy against root-knot nematodes and the potential benefits for plant production.

Abamectin coated (0.3 mg a.i./seed) and non-treated tomato seed (cv. Kirby) was provided by Syngenta Crop Protection. The treatments were seeded in individual seedling trays. After 3 weeks incubation in a greenhouse at 25°±2° C., the seedlings were transplanted into 1500 cm³ pots containing the test soil. The soil was collected from a field at the UC South Coast Research and Extension Center at Irvine (San Emigdio sandy loam, 12.5% sand, 12% clay, 75.4% silt, 0.45 OM, pH 7.4). To improve soil aeration and irrigation water drainage, ⅔ of the soil was mixed with ⅓ (v/v) plaster sand. The soil was pasteurized and infested with root-knot nematodes. Meloidogyne incognita race 3 inoculum was reared on tomato cv. UC 82 for ca. three months in greenhouse cultures. Nematode eggs were harvested from the root systems by a modification of a bleach/sieving method (Hussey and Barker, Plant Disease Reporter, 57:1025-1028 (1973)) and used to infested the test soil with 1000 eggs of M. incognita race 3 per 100 cm³. Pasteuria penetrans was obtained from the University of California Riverside Nematology culture collection. The inoculum was reared on root-knot nematode-infested tomato plants. In the Pasteuria treatments, the soil was amended with approximately 1×10⁵ endospores/g soil. The trial was arranged as a complete randomized block with 6 replications and incubated in a greenhouse at 26°±2° C. with ambient light. All pots were fertilized with Osmocote 14-14-14 (label rate for tomato production). Irrigation was applied as needed. Two months after transplanting, plant tops were cut off at the soil level, oven dried and weighted. Roots were rated for galling on a scale of 0-10 (Zeck, Bayer AG, Pflanzenschutz-Nachrichten, 24:141-144 (1971)). All data were subject to ANOVA and, if appropriate, means separation with Fisher's LSD (SuperANOVA, Abacus, Berkeley, Calif.).

### Results

At the tested infestation level, the root-knot galling in the non-treated check was severe (Table 8). The abamectin seed coating reduced galling by approximately two rating classes which is within the range of typically observed efficacy. The biocontrol agent reduced root galling only slightly. The combination of both abamectin and the biocontrol agent P. penetrans resulted in the lowest gall rating and significantly increased plant top weight compared to the control. Furthermore, it was the only treatment that significantly lowered the root-knot nematode population level at the end of the trial. The results demonstrate the synergistic action by the combined use of the abamectin seed coating and the bacteria.

TABLE 8
Root galling, plant weight and root-knot nematode
population level in soil at trial termination.

| | root gall | plant top dry | J2/50 cc |
|---|---|---|---|
| Treatments | rating (0-10) | weight (g) | soil |
| non-treated check | 5.8 ± 0.5 c | 29.5 ± 2.4 a | 155 ± 75 b |
| abamectin* | 3.3 ± 0.3 ab | 31.3 ± 1.8 ab | 95 ± 15 b |
| P. penetrans** | 5.2 ± 0.4 bc | 33.2 ± 1.2 ab | 135 ± 32 b |
| abamectin* + | 2.3 ± 0.5 a | 36.9 ± 0.6 b | 44 ± 16 a |
| P. penetrans** | | | |

*seed coated (0.3 mg a.i./seed)
**soil incorporated (1 × 10E5/g soil)
means ± standard error; same letter indicate non-significant difference according to Fisher's Protected LSD (0.01)
means ± standard error; same letter indicate non-significant difference according to Fisher's Protected LSD (0.01) after log (x + 1) transformation

All publications and patent applications cited in this specification are herein incorporated by reference as if each individual publication or patent application were specifically and individually indicated to be incorporated by reference.

What is claimed is:

1. A method of treating a plant, the method comprising applying a pesticide composition comprising a nematicide to a plant propagation material, wherein the nematicide is selected from a carbamate nematicide and an avermectin; and applying at least one nematode-antagonistic biocontrol agent selected from Bacillus spp. to the plant propagation material or the planting media of the plant.

2. A method for improving the transplant health of a plant, comprising applying a pesticide composition comprising at least one nematicide to a plant propagation material wherein the nematicide is selected from a carbamate nematicide and an avermectin;
and applying at least one nematode-antagonistic biocontrol agent to the plant propagation material or the planting media of the plant, prior to transplanting the plant, and wherein the biocontrol agent is selected from Bacillus spp.

3. The method of claim 1, wherein the step of applying the pesticide composition to the plant propagation material comprises treating the planting media of the plant with the pesticide composition.

4. The method of claim 1, wherein the step of applying the pesticide composition to the plant propagation material comprises treating the plant propagation material with the pesticide composition.

5. The method of claim 1, wherein the nematicide is thiadicarb and the biocontrol agent is Bacillus Firmus.

6. The method of claim 1, wherein the nematicide is abamectin and the biocontrol agent is Bacillus Firmus.

7. The method according to claim 1, wherein the plant propagation material is a seed.

8. The method according to claim 1, wherein the step of applying the at least one biocontrol agent comprises treating the plant propagation material with the at least one biocontrol agent prior to planting.

9. The method according to claim 1, wherein the step of applying the at least one biocontrol agent comprises inoculating the planting media of the plant with the at least one biocontrol agent.

10. The method of claim 9, wherein the step of inoculating the planting media with the at least one biocontrol agent is performed prior to planting the plant propagation material.

11. The method of claim 9, wherein the step of inoculating the planting media with the biocontrol agent is performed while planting the plant propagation material.

12. A combination composition comprising a pesticide control agent comprising an effective amount of at least one nematicide selected from a carbamate nematicide and an avermectin and an effective amount of at least one biocontrol agent selected from Bacillus spp.

13. The combination composition of claim 12, wherein the nematicide is thiadicarb and the biocontrol agent is Bacillus Firmus.

14. The combination composition of claim 13, wherein the nematicide is abamectin and the biocontrol agent is Bacillus Firmus.

15. The combination composition according claim 12, wherein the at least one biocontrol agent is an nematode-antagonistic biocontrol agent.

16. The combination composition according to claim 12, further comprising a seed or plant to which the pesticide control agent and biocontrol agent have been applied.

17. The combination composition of claim 24, further comprising planting media, wherein the composition is contained within a container.

18. The combination composition according to claim 12, wherein the pesticide composition comprises at least one additional insecticide, nematicide, acaricide or molluscicide.

19. The combination composition according to claim 12, wherein the pesticide composition further comprises at least one additional fungicide.

20. Plant propagation material treated with the combination composition according to claim 12.
